(19) United States
(12) Patent Application Publication
(10) Pub. No.: US 2022/0096511 A1
(43) Pub. Date: Mar. 31, 2022

(54) ANTIFUNGAL AGENTS WITH IMPROVED WATER SOLUBILITY

(71) Applicant: Sigma-Aldrich Co. LLC, St. Louis, MO (US)

(72) Inventors: Aviran Amir, Rosh Haayn (IL); Tamar Dvash, Kibbutz Tzuba (IL); Michal Levin-Khalifa, Jerusalem (IL); Theo Ackermann, Jerusalem (IL)

(21) Appl. No.: 17/426,934

(22) PCT Filed: Jan. 31, 2020
(86) PCT No.: PCT/US2020/016165

§ 371 Date: Jul. 29, 2021

Related U.S. Application Data
(60) Provisional application No. 62/799,442, filed on Jan. 31, 2019.

(51) Int. Cl. A61K 31/7048; C07H 17/08; A61P 31/10

(52) U.S. Cl. CPC A61K 31/7048; A61P 31/10; C07H 17/08

(57) ABSTRACT

Methods for preparing a polyene macrolide antifungal with improved aqueous solubility. The method involves providing a polyene macrolide antifungal having a carboxylic acid group; activating the carboxylic acid group; introducing a primary amine to the activated polyene macrolide antifungal; reacting for a time sufficient to convert the carboxylic acid to an amide, and quenching the reaction, thus yielding a polyene macrolide amide or salt thereof. Also provided are water-soluble polyene macrolide derivatives.

### CROSS-REFERENCE TO RELATED APPLICATIONS

This application claims the benefit of priority to U.S. Provisional Patent Application No. 62/799,442, filed Jan. 31, 2019, the entirety of which is incorporated herein by reference.

### BACKGROUND

Fungal infections are a worldwide threat that affects people with varied severity from skin rash to mortality. The most severe illnesses arising from invasive candidiasis and aspergillosis have higher mortality rates as compared to bacterial infections. Furthermore, immunocompromised people are the most at risk and in recent reports fungal infections have been recognized as an enraging threat for intensive care unit (ICU) patients.

The most common fungal infections are associated with Candida or Aspergillus. Candida sp. is a yeast known to cause several diseases such as: thrush, vaginal and invasive candidiasis. In most cases the pathogenic species is C. albicans, C. parapsilosis and C. glabrata. Aspergillus is a mold which is less common then Candida in causing invasive infections. However, invasive aspergillosis is a life-threatening illness and highly misdiagnosed causing mortality in ICU units.

In the recent years, antifungal resistant fungi have emerged. C. auris, is one example of a life-threatening fungus that has been found in hospitalized patients. Most C. auris isolates were found to be resist to fluconazole and about third were resist to amphotericin.

Polyene macrolide antifungal agents are a group of small molecules, including nystatin, amphotericin B, and natamycin, which are commonly used in clinic and for research. Polyene macrolide antifungal agents are water insoluble and therefore the bioavailability of these compounds in aqueous-based systems is reduced. This generates a major challenge to introduce them, for instance, to live cell culture where media is an aqueous solution.

Currently available nystatins are used either solubilized in DMSO/DMF or suspended in aqueous media. A need for water soluble forms of nystatin or other polyene macrolide antifungal agents has long been recognized, however, no adequate solutions have been found so far.

Several methods have employed to try to increase the solubility of polyene macrolide antifungal agents in aqueous solutions with varying degrees of success. Aside from just increasing solubility in aqueous media, the potency of the compounds must be maintained, while maintaining safety of the formulation for use in, e.g., cell culture media or even clinical applications.

One method that has been used to deliver polyene macrolide antifungal agents within aqueous systems is liposomal encapsulation, though the results using this method have been mixed. See, e.g., Johnson et al., Antimicrobial Agents and Chemotherapy, Vol. 42, No. 6, June 1998. Other methods that have been tried with some success. GB809105A describes a method for preparing polysaccharide conjugates of polyene antibiotic to increase solubility in water. U.S. Pat. No. 4,783,527A describes amide derivatives of various antibiotics and their derivatives but does not suggest modifications for nystatin. US20090186838A1 and WO0191758A1 each describe water-soluble amide derivatives of amphotericin; however, neither ethanol amine nor ethanol amide derivatives or methods of making those are taught. WO2001051061A1 describes water soluble glycosyl derivatives of polyene macrolides. WO2013132014 describes the use of sterically hindered derivatives of the antifungal antibiotic, Nystatin A1 which contain bulky fragments on the substituent linked to the amino group of the antibiotic. However, none of these have led to a suitable, widely adopted, solution for use of nystatin or other water-insoluble polyene macrolide antifungal agents in aqueous media.

In spite of these and other previous attempts to provide an effective, safe, water-soluble form of nystatin and other polyene macrolide antifungal agents, a need still exists for improved water-soluble antifungal agents for use in aqueous-based systems such as cell culture media.

### SUMMARY

Provided herein are improved methods for preparing a polyene macrolide antifungal with improved aqueous solubility. The method involves providing a polyene macrolide antifungal having a carboxylic acid group; activating the carboxylic acid group; introducing a primary amine to the activated polyene macrolide antifungal; reacting for a time sufficient to convert the carboxylic acid to an amide, and quenching the reaction, thus yielding a polyene macrolide amide or salt thereof.

In various embodiments, the polyene macrolide antifungal may be nystatin, amphotericin, candicidin, natamycin, polyfungin, or Levorin. In a particularly preferred embodiment, the polyene macrolide antifungal is nystatin.

The primary amine used in the methods provided may be unsubstituted or substituted C₁-C₁₀ alkylamines, unsubstituted or substituted C₁-C₁₀ alcoholamines, unsubstituted or substituted amino acids, or hydroxylamine. In various embodiments described herein the primary amine may be ethanolamine, lysine, hydroxylamine, leucenol, methylamine, ethylamine, propylamine or butylamine. In a particularly preferred embodiment, the primary amine is ethanolamine.

In embodiments in which the method is used to yield a salt, the polyene macrolide amide salt includes a counterion such as acetate, formate, propionate, butyrate, chloride or sulfate.

In some embodiments, the method further includes the step of separating the resulting polyene macrolide amide or salt thereof to provide an isolated polyene macrolide amide or salt thereof.

Further provided are compounds of Formula I

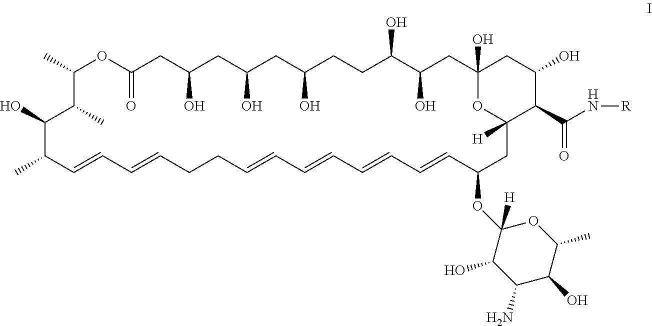

wherein R is a selected from the group consisting of C₁-C₁₀ alkyl, C₁-C₁₀ substituted alkyl, C₁-C₁₀ alcohol, C₁-C₁₀ substituted alcohol, and hydroxyl, wherein the substituents are selected from the group consisting of alcohols, amines, carboxylic acids and salt thereof. In a particularly preferred embodiment, R is ethanol.

In some embodiments, the compound of Formula I may be a salt. In these embodiments, it further includes a counterion selected from, e.g., acetate, formate, propionate, butyrate, chloride and sulfate.

In a particularly preferred embodiment, the compound is nystatin ethanol amide having the structure:

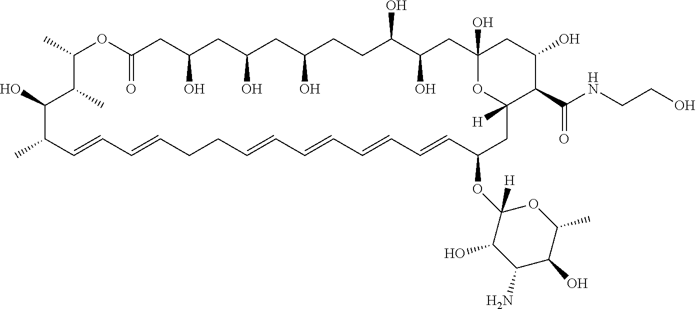 [SMILES: C[C@@H]1[C@H](O)[C@@H](C)/C=C/C=C/CC/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C(=O)NCCO]

or a salt thereof.

Further provided are compositions for the treatment of a fungal infection in a subject, the compositions including a pharmacologically effective amount of a compound of Formula I or a pharmaceutically acceptable salt thereof.

### BRIEF DESCRIPTIONS OF THE DRAWINGS

FIG. 1 shows the structure of a preferred nystatin ethanol amide described herein.

FIG. 2 shows (A) shows a mass spectrum of a nystatin ethanol amide described herein; (B) shows the m/z region from 940 to 1020.

FIG. 3 shows a UV/Vis spectrum of an exemplary nystatin ethanol amide prepared using the method described herein.

FIG. 4 is an HPLC chromatogram of an exemplary nystatin ethanol amide prepared using the method described herein.

FIG. 5 is a photo showing the results of potency testing according to method USP/81 of an exemplary nystatin ethanol amide prepared using the method described herein.

FIG. 6 is a proton NMR of nystatin ethanol amide in D₂O.

FIG. 7 is a 2D NMR of nystatin ethanol amide in D₂O.

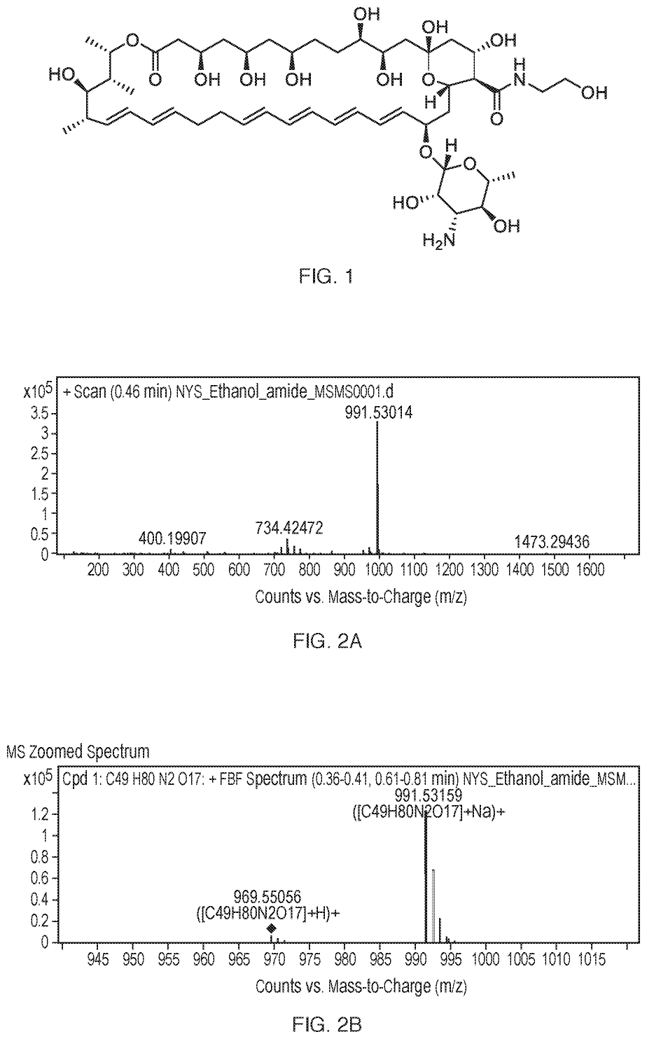

### DETAILED DESCRIPTION

Provided herein are methods of preparing water-soluble derivatives of polyene macrolide antifungal agents having improved bioavailability in aqueous systems and the antifungal agents prepared by these methods. The modifications described herein allow for polyene macrolides with enhanced water solubility without detrimental effect on the antifungal activity of the compound. The compounds prepared by the methods described herein are suitable for research purposes and potential clinical use.

As used herein the term “antifungal agent” refers generally to polyene macrolide antifungal agents, sometimes referred to herein simply as polyene macrolide antifungals, antifungals or antifungal agents. Antifungal agents suitable for methods described herein include, for example, nystatin, amphotericin B (also referred to as “amphotericin”), candicidin, natamycin, polyfungin, and Levorin.

The terms “antifungal activity” or “potency,” these terms being used interchangeably, refer to the inhibitory effect of an antifungal agent on microorganisms under suitable conditions as measured using a standard analytical method, such as the methods established by the United States Pharmacopeial Convention (USP).

As used herein, the term “bioavailability” refers to the proportion of antifungal agent solubilized in aqueous solution and thus able to have an active effect on microorganisms it comes into contact with.

“Improved bioavailability,” as used herein, means that the bioavailability of antifungal agent is improved when compared with the same amount of antifungal agent in a convention preparation, such as in DMSO/DMF or suspended in an aqueous medium.

The methods for preparing a polyene macrolide antifungal with improved aqueous solubility, involve providing a polyene macrolide antifungal having a carboxylic acid group; activating the carboxylic acid group; introducing a primary amine to the activated polyene macrolide antifungal; reacting for a time sufficient to convert the carboxylic acid to an amide, and quenching the reaction, thus yielding a polyene macrolide amide or salt thereof.

In various embodiments, the polyene macrolide antifungal may be nystatin, amphotericin, candicidin, natamycin, polyfungin, or Levorin. In a particularly preferred embodiment, the polyene macrolide antifungal is nystatin.

In a preferred embodiment, a coupling reagent, such as HCTU, is used to activate the carboxylic acid group.

The primary amine may be an unsubstituted or substituted C₁-C₁₀ alkylamine, an unsubstituted or substituted C₁-C₁₀ alcoholamine, an unsubstituted or substituted amino acid, or hydroxylamine. When the primary amine is substituted, it may be substituted at any substitutable position with one or more substituents selected from the group consisting of alcohols, amines, carboxylic acids and combinations thereof. In various embodiments described herein the primary amine may be ethanolamine, lysine, hydroxylamine, leucenol, methylamine, ethylamine, propylamine or butylamine. In preferred embodiments, the primary amine is selected from ethanolamine, lysine, hydroxylamine and leucenol. In a particularly preferred embodiment, the primary amine is ethanolamine.

In embodiments in which the method is used to yield a salt, the polyene macrolide amide salt includes a counterion; in preferred embodiments, the counterion may be acetate, formate, propionate, butyrate, chloride or sulfate.

In some embodiments, the method further includes the step of separating the resulting polyene macrolide amide or salt thereof to provide an isolated polyene macrolide amide or salt thereof.

Further provided are water-soluble antifungal compounds of Formula I

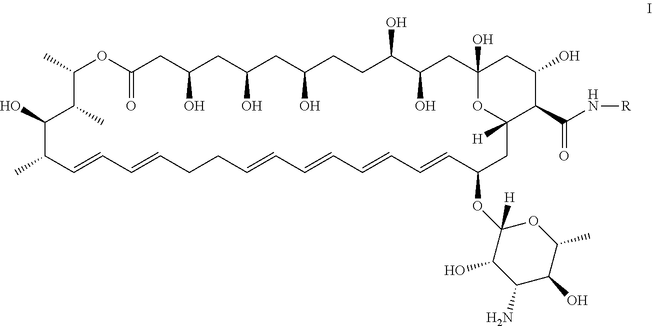

wherein R is selected from the group consisting of C₁-C₁₀ alkyl, C₁-C₁₀ substituted alkyl, C₁-C₁₀ alcohol, C₁-C₁₀ substituted alcohol, and hydroxyl, wherein the substituents are selected from the group consisting of alcohols, amines, carboxylic acids and salt thereof. In some embodiments, R may be an amino acid or a derivative of an amino acid.

In some embodiments, R is selected from the group consisting of C₁-C₆ alkyl, C₁-C₆ substituted alkyl, C₁-C₆ alcohol, C₁-C₆ substituted alcohol, and hydroxyl. In still other embodiments, R is selected from the group consisting of C₁-C₃ alkyl, C₁-C₃ substituted alkyl, C₁-C₃ alcohol, C₁-C₃ substituted alcohol, and hydroxyl.

In embodiments in which R includes one or more substituents, the substituents may be selected from one or more of, e.g., C₁-C₆ alkyl, C₁-C₆ substituted alkyl, C₁-C₆ alcohol, C₁-C₆ substituted alcohol or a combination thereof.

In still other embodiments, R includes one or more substituents, the substituents may be selected from one or more of, e.g., C₁-C₃ alkyl, C₁-C₃ substituted alkyl, C₁-C₃ alcohol, C₁-C₃ substituted alcohol or a combination thereof.

In a particularly preferred embodiment, R is ethanol.

In some embodiments, the compound of Formula I may be a salt. In these embodiments, it further includes a counterion selected from, e.g., acetate, formate, propionate, butyrate, chloride and sulfate.

In a particularly preferred embodiment, the compound is nystatin ethanol amide having the structure:

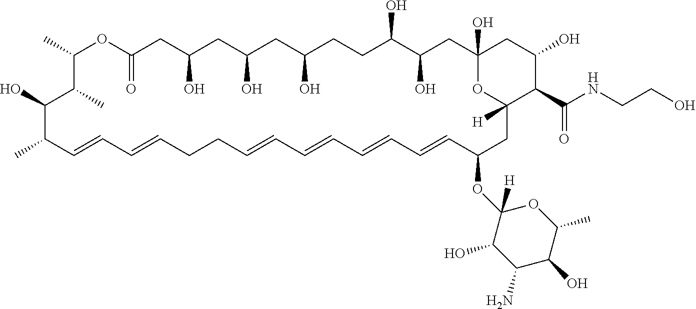 [SMILES: C[C@@H]1[C@H](O)[C@@H](C)/C=C/C=C/CC/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C(=O)NCCO]

or a salt thereof.

The salt form is shown in Formula IA

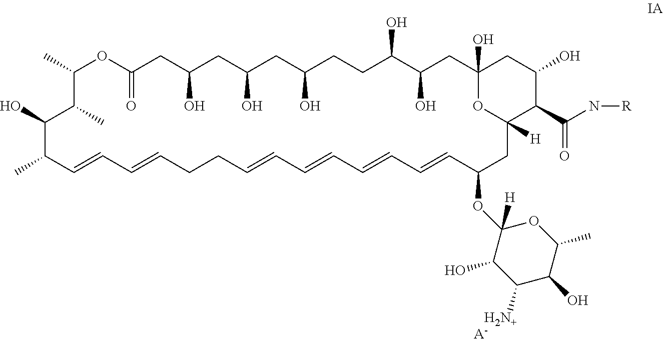

wherein the anion, A⁻, is selected from the group consisting of acetate, formate, propionate, butyrate, chloride, sulfate or combinations thereof. R is as defined above.

Method of Preparing the Water-Soluble Derivative

A polyene macrolide antifungal agent is selected and dissolved in dry DMF. A primary amine, as described herein, is added to the solution followed by the addition of a coupling agent, such as HCTU. The mixture is allowed to react for a time sufficient to allow the reaction to complete. The reaction is then quenched, and optionally, converted to the salt form. The resulting product can then be purified and optionally, freeze dried.

The method outlined above can be modified for various polyene macrolide antifungal agents, such as nystatin, amphotericin, candicidin, natamycin, polyfungin and Levorin, as well as other polyene macrolides.

The amine may be any of a variety of unsubstituted or substituted primary amines, including unsubstituted or substituted C₁-C₁₀ alkylamine, unsubstituted or substituted C₁-C₁₀ alcoholamine, unsubstituted or substituted amino acids, or hydroxylamine. When the primary amine is substituted, it may be substituted at any substitutable position with one or more substituents selected from the group consisting of alcohols, amines, carboxylic acids and combinations thereof. In various embodiments described herein the primary amine may be ethanolamine, lysine, hydroxylamine, leucenol, methylamine, ethylamine, propylamine or butylamine. In preferred embodiments, the primary amine is selected from ethanolamine, lysine, hydroxylamine and leucenol. In a particularly preferred embodiment, the primary amine is ethanolamine.

Potency Testing

Potency testing is done using a cylinder plate assay using the methods described in U.S. Pharmacopeia, e.g., Pharmacopeial Forum, Vol. 36(6) [November-December 2010] <81> Antibiotics—Microbial Assays, USP 32 page 86 ff.

Uses for the Water-Soluble Antifungal Agents

Because of their improved water solubility, the polyene macrolide antifungal derivatives described herein have improved bioavailability over their non-derivatized counterparts. The improved bioavailability allows for reduced dosages, and therefore reduced toxicity. The higher bioavailability also allows for antifungal applications that have not been previously realized due to low solubility.

Additional applications for the water-soluble polyene macrolide antifungal agents described herein include additional clinical applications for human and veterinary use. These include topical treatments of fungal infection for dermatological infections; improved formulations for treatment of oral infections and vaginal infections, injectable and/or parenteral forms for systemic infection, such as fungal superinfection in the respiratory tract during bacteremia/sepsis, and following transplantation.

The compositions for treatment of fungal infections may include pharmaceutically acceptable additives. For example, for oral administration, such pharmaceutically acceptable additives may include excipients, such as binding agents, such as pregelatinised maize starch, polyvinylpyrrolidone or hydroxypropyl methylcellulose; fillers, such as lactose, microcrystalline cellulose or calcium hydrogen phosphate; lubricants, such as magnesium stearate, talc or silica; disintegrants, such as starch or starch derivatives; surfactants; or coatings. Liquid preparations may be prepared with pharmaceutically acceptable additives including, for example, suspending agents such as sorbitol syrup, cellulose derivatives or hydrogenated edible fats; emulsifying agents such as lecithin or acacia; preservatives, such as methyl or propyl-p-hydroxybenzoates or sorbic acid; buffer salts, flavoring, coloring and sweetening agents as appropriate. Preparations for oral administration may be suitably formulated to give controlled release of the active compound.

In other embodiments, the water-soluble antifungal agents described herein may be formulated in compositions for parenteral administration, e.g., for injection, by bolus injection or continuous infusion. Formulations for injection may be presented in unit dosage form, e.g., in ampoules or in multidose containers, with an added preservative if desired. The compositions may take such forms as suspensions, solutions or emulsions in aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Topical preparations may be in the form of lotions, creams or ointments. Ointments may include a carrier, such as soft paraffin or white petrolatum, and other desired additives such as surfactants; solvents; excipients; preservatives, such as benzoic acid; emulsifying agents, such as polysorbates, e.g. polysorbate 60; and viscosity enhancing agents, such as cetostearyl alcohol, along with the water-soluble antifungal agent described herein. Creams include the oily phase of an ointment as a melt as described above, combined with suitable oil and water-soluble surfactants and an aqueous phase containing the drug and suitable antimicrobial preservatives.

Other suitable formulations including the water-soluble antifungal agents described herein for administration topically, orally, vaginally, or parenterally can be readily determined by one skilled in the art.

Further provided herein are methods for treating fungal infections in a subject, the methods including the step of administering a pharmacologically effective amount of a water-soluble polyene macrolide antifungal agent provided herein to a subject in need of such treatment. In accordance with the method, the subject may be a human subject, or the subject may be an animal, i.e., in veterinary applications.

### Examples

Nystatin ethanol amide is produced as follows:

8.73 g Nystatin (Sigma-Aldrich, St. Louis, Mo.) was dissolved in 260 ml dry Dimethylformamide (DMF). 5.2 ml ethanolamine was added followed by addition of 8.73 g HCTU (Sigma-Aldrich, St. Louis, Mo.). The mixture was allowed to react, then another portion of HCTU was added and the mixture was allowed to react for additional time. HPLC indicated almost complete conversion. The reaction mixture was poured into 2.5 L ethyl acetate and the product precipitated. The mixture was decanted and the resulted oily solid dissolved in methanol and purified by reverse phase chromatography. The purified fractions were pooled and freeze dried. The yellowish solid was dissolved in 250 mL water and freeze dried. The freeze dried solid was dissolved in 50 mL ethanol and filtered over 0.2-μm membrane, 250 mL water was added and the mixture was freeze dried to give 1.93 g, 20% yield of the Nystatin ethanol amide (acetic acid salt).

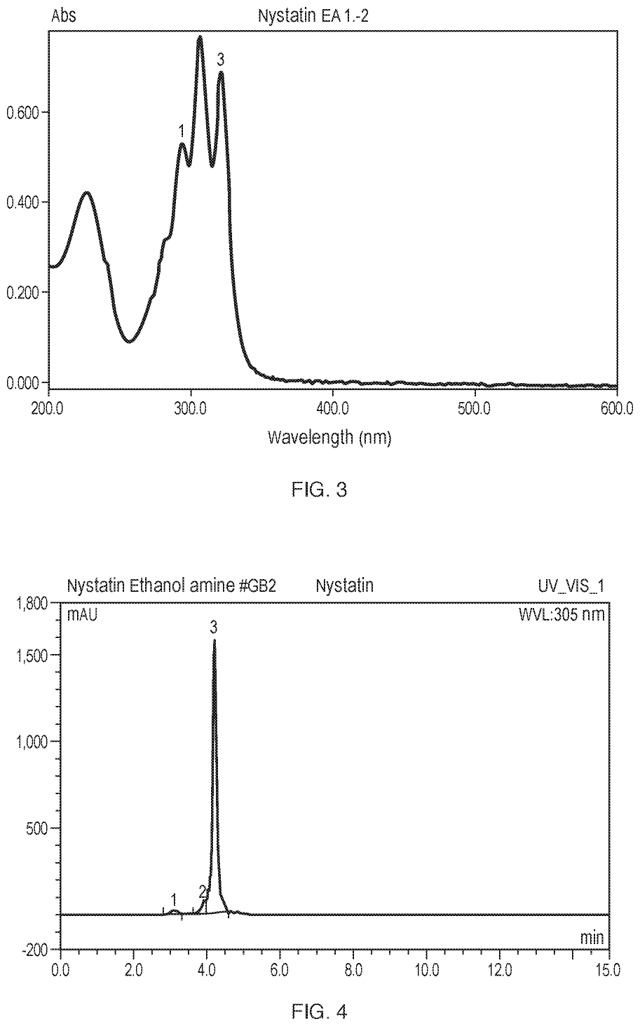

A mass spectrum of a nystatin ethanol amide is shown in FIG. 2A, FIG. 2B shows the m/z region from 940 to 1020. This confirms the formation of the nystatin ethanol amide. FIG. 3 shows a UV/Vis spectrum of the nystatin ethanol amide. Purity of the product was confirmed by HPLC as shown in FIG. 4.

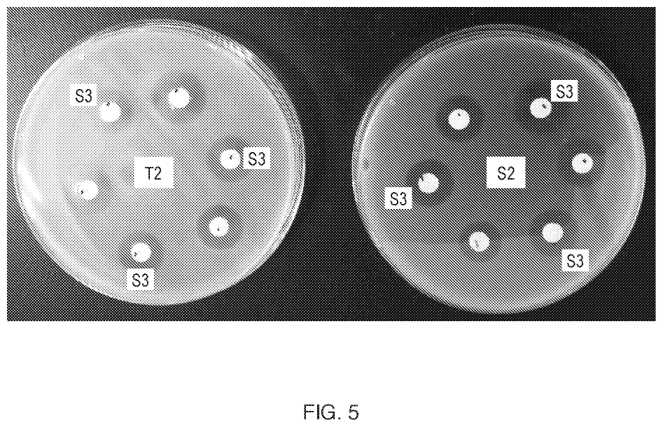

FIG. 5 is a photo showing the results of potency testing according to method USP/81 of the nystatin ethanol amide product. The potency for 1 mg nystatin ethanol amide was 5500-6500 U.

Endotoxin testing (LAL) was performed using standard methods and found to be <30 Eu/mg.

Toxicity: Toxicity was tested with hep2 cell line by checking their viability after incubation with the compound. The amount of the compound that corresponds to 50% cell viability is the maximum amount of compound that can be apply on to cell culture. In that case the nystatin ethanol amide was the same or better than the unsalable nystatin.

Minimum inhibitory concentration (MIC) testing was determined for nystatin ethanol amide along with comparative antifungal agents.

Nystatin (Solid) and amphotericin were dissolved in DMF in 1 mg/mL. Econazole nitrate and Nystatin ethanol amide (salt) were dissolved in PBS to achieve 1 mg/mL.

Antibiotic medium 19 was sterilized in autoclave, after sterilization when the temperature lowered to 45° C. and 1 mL from spore suspension was added. 8 mL from this mixture dispensed to each petri dish.

On each dish 6 sterile diffusion paper disc were placed, 10 μL from each antibiotic were applied in 10, 15, 20, 25, 30, 35 μg/mL concentrations. Where MIC concentrations were below 10, lower concentrations were used: 2, 5, 7.5 μg/mL

The petri dishes were incubated overnight at 30° C. The MIC were determined as the lower concentration that inhibition could be visualized. The results are summarized in Table 1, below.

TABLE 1
Inhibition of Candida albicans and Aspergillus niger
in μg/mL, each result is an average of three experiments.

| Antifungal agent: | Candida albicans | Aspergillus niger |
|---|---|---|
| Amphotericin | 16 | 31.5 |
| Econazole | No inhibition | No inhibition |
| Nystatin | 14 | 22.5 |
| Nystatin ethanol amide | 15.7 | 23.6 |
| Polymixin B | No inhibition | No inhibition |
| Nystatin Methyl ester | 30 | No inhibition |

Observations. In this diffusion disc assay we measured the minimal concentration applied by each compound needed to inhibit the growth of C. albicans and A. niger. Nystatin ethanol amide inhibited the growth of C. albicans (15.7 μg/mL) and A. niger (23.6 μg/mL) similarly to Nysatin 14 μg/mL and 22.5 μg/mL respectively. Interestingly, Nystatin methyl ester, which is the Nystatin and Nystatin ethanol amide analog, was found to be less active (30 μg/mL) against C. albicans and with no observed inhibition for A. niger. Amphotericin B inhibit the growth of C. albicans (16 μg/mL) similarly to Nystatin and Nystatin water soluble. However, Amphotericin B was less active (31.5 μg/mL) in inhibiting the growth of A. niger.

The examples provided herein are illustrative in nature and are not meant to limit the scope of the invention as set forth within the claims.

The invention claimed is:

1. A method for preparing a polyene macrolide antifungal with improved aqueous solubility, the method comprising
providing a polyene macrolide antifungal having a carboxylic acid group;
activating the carboxylic acid group;
introducing a primary amine to the activated polyene macrolide antifungal;
reacting for a time sufficient to convert the carboxylic acid to an amide, and
quenching the reaction, wherein the resulting product is a polyene macrolide amide or salt thereof.

2. The method of claim 1 wherein the polyene macrolide antifungal is selected from the group consisting of nystatin, amphotericin, candicidin, natamycin, polyfungin, and Levorin.

3. The method of claim 2 wherein the polyene macrolide antifungal is nystatin.

4. The method of claim 1 wherein the carboxylic acid group is activated with a coupling reagent.

5. The method of claim 4 wherein the coupling reagent comprises HCTU.

6. The method of claim 1 wherein the primary amine is selected from the group consisting of optionally substituted C₁-C₁₀ alkylamine, optionally substituted C₁-C₁₀ alcoholamine, amino acids, and hydroxylamine, wherein the optional substituent, if present, is selected from the group consisting of alcohols, amines, carboxylic acids and combinations thereof.

7. The method of claim 6, wherein the primary amine is selected from the group consisting of ethanolamine, lysine, hydroxylamine, leucenol, methylamine, ethylamine, propylamine, butylamine, and combinations thereof.

8. The method of claim 7 wherein the primary amine is selected from the group consisting of ethanolamine, lysine, hydroxylamine and leucenol.

9. The method of claim 8 wherein the primary amine is ethanolamine.

10. The method of claim 1 wherein the polyene macrolide amide salt includes a counterion selected from the group consisting of acetate, formate, propionate, butyrate, chloride and sulfate.

11. The method of claim 1 further comprising the step of separating the resulting polyene macrolide amide or salt thereof to provide an isolated polyene macrolide amide or salt thereof.

12. A compound of Formula I 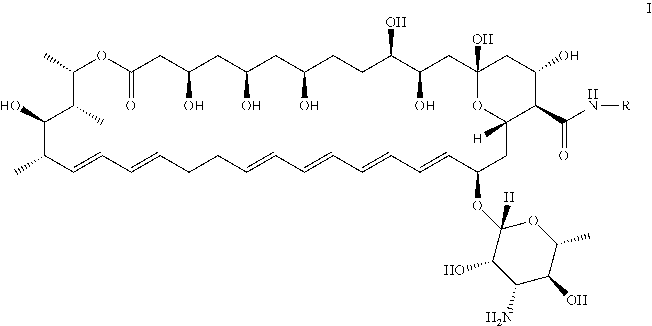
wherein R is a selected from the group consisting of C₁-C₁₀ alkyl, C₁-C₁₀ substituted alkyl, C₁-C₁₀ alcohol, C₁-C₁₀ substituted alcohol, and hydroxyl, wherein the substituents are selected from the group consisting of alcohols, amines, carboxylic acids and combinations thereof,
or a salt thereof.

13. The compound of claim 12, wherein R is selected from the group consisting of C₁-C₆ alkyl, C₁-C₆ substituted alkyl, C₁-C₆ alcohol, C₁-C₆ substituted alcohol or a combination thereof.

14. The compound of claim 13 wherein R is ethanol.

15. The compound of claim 12 wherein the compound of Formula I comprises a salt, wherein the counterion is selected from the group consisting of salt includes a counterion selected from the group consisting of acetate, formate, propionate, butyrate, chloride and sulfate.

16. Nystatin ethanol amide having the structure: 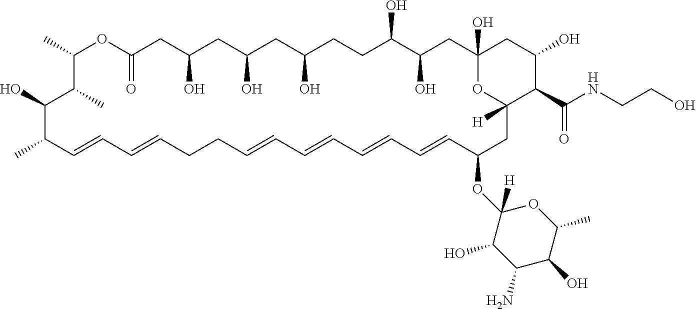 [SMILES: C[C@@H]1[C@H](O)[C@@H](C)/C=C/C=C/CC/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C(=O)NCCO]
or a salt thereof.

17. A composition for the treatment of a fungal infection in a subject, the composition comprising a compound of Formula I 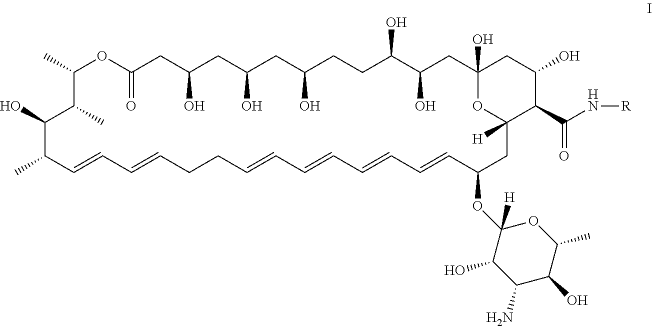
wherein R is a selected from the group consisting of C₁-C₁₀ alkyl, C₁-C₁₀ substituted alkyl, C₁-C₁₀ alcohol, C₁-C₁₀ substituted alcohol, and hydroxyl, wherein the substituents are selected from the group consisting of alcohols, amines, carboxylic acids and combinations thereof,
or a salt thereof.

18. A method for the treatment of a fungal infection in a subject, the method comprising the step of administering a pharmacologically acceptable amount of a compound of Formula I 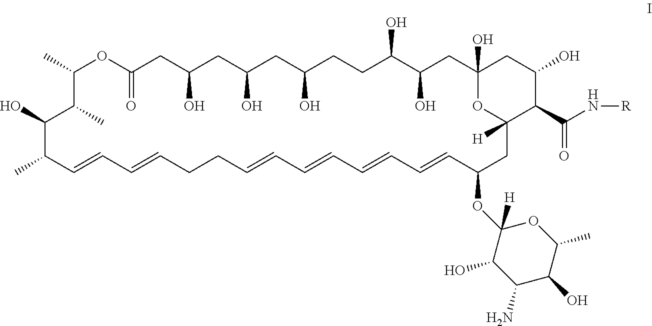
wherein R is a selected from the group consisting of C₁-C₁₀ alkyl, C₁-C₁₀ substituted alkyl, C₁-C₁₀ alcohol, C₁-C₁₀ substituted alcohol, and hydroxyl, wherein the substituents are selected from the group consisting of alcohols, amines, carboxylic acids and combinations thereof,
or a salt thereof
to a subject in need of such treatment.

19. The method of claim 18 wherein the compound of Formula I is 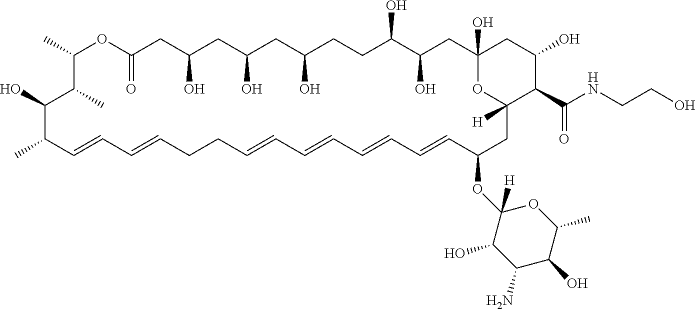 [SMILES: C[C@@H]1[C@H](O)[C@@H](C)/C=C/C=C/CC/C=C/C=C/C=C/C=C/[C@H](O[C@@H]2O[C@H](C)[C@@H](O)[C@H](N)[C@@H]2O)C[C@@H]2O[C@](O)(C[C@@H](O)[C@H](O)CC[C@@H](O)C[C@@H](O)C[C@@H](O)CC(=O)O[C@H]1C)C[C@H](O)[C@H]2C(=O)NCCO]
or a pharmaceutically acceptable salt thereof.

20. The method of claim 18 wherein the fungal infection is associated with Candida or Aspergillus.
